(19) United States
(12) Patent Application Publication
(10) Pub. No.: US 2016/0220127 A1
(43) Pub. Date: Aug. 4, 2016

(54) WELLNESS OR ILLNESS ASSESSMENT SYSTEM, METHOD, AND COMPUTER PROGRAM PRODUCT

(71) Applicant: Covidien LP, Mansfield, MA (US)

(72) Inventor: Robert T. Boyer, Longmont, CO (US)

(21) Appl. No.: 14/612,931

(22) Filed: Feb. 3, 2015

(51) Int. Cl. A61B 5/0205; A61B 5/00

(52) U.S. Cl. CPC A61B 5/02055; A61B 5/7278; A61B 5/0022; A61B 5/742; A61B 5/024

(57) ABSTRACT

Methods and systems are provided for assessing and forecasting a wellness or illness of a patient. A system includes a mobile device including a processor, a device memory in communication with the processor, and a display in communication with the processor. The device memory includes instructions that, when executed, cause the processor to receive a plurality of patient parameters, the plurality of patient parameters including one or more of a temperature of the patient, a pulse of the patient, a respiration rate of the patient, and a blood pressure of the patient, receive a manual input of an additional value corresponding to a subjective parameter of the patient, compare the plurality of patient parameters and the additional value with baseline data, assign a wellness rating to the patient, based on the comparison, and display recommended courses of action associated with the wellness rating of the patient.

### TECHNICAL FIELD

The present disclosure relates generally to patient monitoring and to systems and methods for assessing a wellness or illness of a patient.

### BACKGROUND

Determining whether a patient is too sick to attend school or work can be a relatively difficult task. Typically, a parent or other caretaker of the patient conducts a general visual examination of the patient's physical symptoms to determine a severity of illness. Additionally, the caretaker may take the patient's temperature, either by use of a thermometer or a hand to the forehead. In instances in which the patient does not have a caretaker, the patient may evaluate his or her overall health condition with or without taking a temperature.

### SUMMARY

The present disclosure relates to methods and systems for assessing and forecasting a wellness or illness of a patient. In accordance with aspects of the present disclosure, a system is disclosed including a mobile device including a processor, a device memory in communication with the processor, and a display in communication with the processor. The device memory includes instructions that, when executed, cause the processor to receive a plurality of patient parameters, the plurality of patient parameters including one or more of a temperature of the patient, a pulse of the patient, a respiration rate of the patient, and a blood pressure of the patient, receive a manual input of an additional value corresponding to a subjective parameter of the patient, compare the plurality of patient parameters and the additional value with baseline data, assign a wellness rating to the patient, based on the comparison, and display recommended courses of action associated with the wellness rating of the patient.

The present disclosure provides new and unique ways to more accurately assess a patient's condition in order to determine whether the patient is healthy or sick. By collecting real-time parameter data from the patient and comparing the collected data with the patient's or other patients' historical data, the comparison results may be associated with popular courses of actions taken by the other patients. Conveying the courses of action to the patient may allow the patient to make a more informed decision as to whether to attend school or work, to stay home, or to visit a nurse or doctor. As a result, the patient may be prevented from taking unnecessary sick days, or the number of instances in which the patient may be sent home later in the day may be reduced.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

### BRIEF DESCRIPTION OF THE DRAWINGS

The present disclosure and its various aspects and features are described hereinbelow with reference to the accompanying drawings, wherein:

FIG. 1 is a schematic illustration of a system for assessing a wellness or illness of a patient provided in accordance with the present disclosure;

FIG. 2 is a schematic illustration of a hardware and software configuration for use with the system of FIG. 1;

FIG. 3 illustrates a display screen provided in accordance with the present disclosure, as presented to a user;

FIG. 4 illustrates another display screen provided in accordance with the present disclosure, as presented to a user;

FIG. 5 illustrates another display screen provided in accordance with the present disclosure, as presented to a user;

FIG. 6 illustrates another display screen provided in accordance with the present disclosure, as presented to a user;

FIG. 7 is a flow diagram illustrating one implementation of the present disclosure; and

FIGS. 8A-8C are bar graphs provided in accordance with the present disclosure, as presented to a user.

### DETAILED DESCRIPTION

Provided in accordance with the present disclosure are systems and methods for assessing a wellness or illness of a patient. The assessment of wellness or illness is based on parameters, such as vital sign data and subjective observation data. The vital sign data are collected by one or more sensors placed adjacent or on the patient and are transmitted either wirelessly or via cables to a mobile device. The sensors may be lightweight and portable and may be separate components from the mobile device. The sensors may attach to or detach from the mobile device. The mobile device may be a smart phone, tablet, or other device on which an application for assessing the developing illness of the patient may be downloaded and executed. The application provides the user with interactive screens that guide the user through step-by-step instructions in a clear and concise manner. Due to its portability, the system may be used in a wide variety of settings, such as, in a home, school, work, clinic, urgent care or hospital facility, or any other setting in which the patient may be located.

As used herein, the term “user” includes, but is not limited to, a parent, guardian, doctor, nurse, other medical personnel, any other caregiver, or the patient.

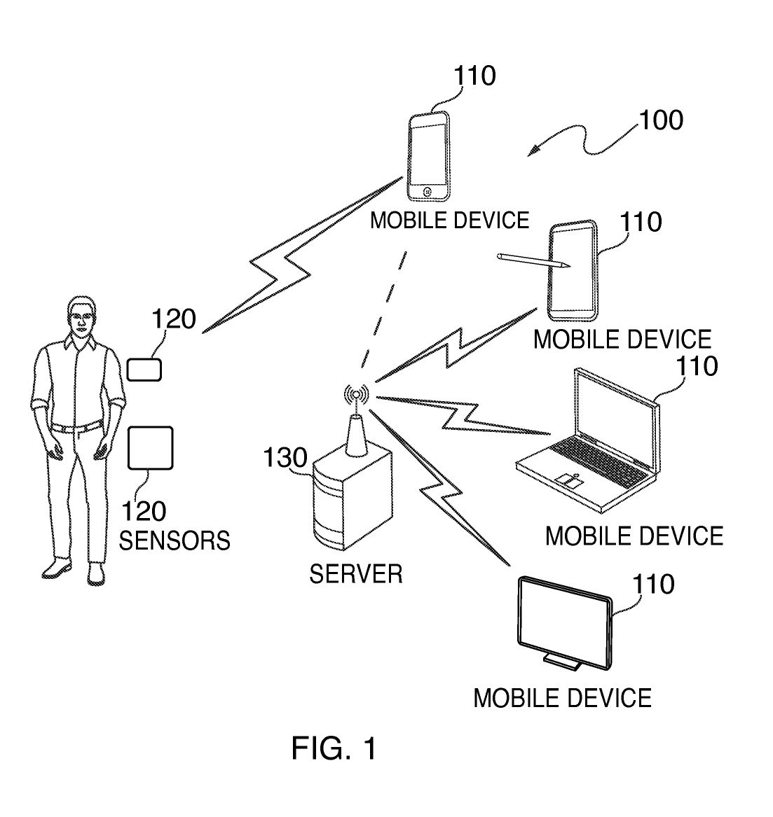

Referring to FIG. 1, an exemplary system provided in accordance with the present disclosure is shown generally identified by reference numeral 100. System 100 includes a mobile device 110, one or more sensors 120, and one or more servers 130. For the purposes herein, exemplary system 100 is generally described, although the aspects and features of the present disclosure may be implemented, incorporated, or utilized with any other devices, systems, and combinations thereof.

Mobile device 110 requests and receives information relating to the patient, such as parameters, conditions, sensed data, and/or other information, and processes and displays the patient information to a user. The information may be displayed via a display, user interface, browser, and/or application running on the mobile device 110. Alternatively, mobile device 110 outputs the information to the user, e.g., prints a generated report containing the information.

Mobile device 110 may further be configured to receive input from the user. For example, mobile device 110 may include a touchscreen, buttons, a keyboard, mouse, and the like, configured to be manually manipulated by the user, to input information such as data or to input commands such as controlling the display or output of the information, the setting of parameters, the resetting notifications, and the like. Information input into mobile device 110 may include medical care information, for example, health care provider information (e.g., doctor, hospital, or school nurse contact information, health insurance information, etc.), prescription information, control parameters, other measured data, and/or biographical or observed data.

Mobile device 110 may be a single device or may include multiple devices and may incorporate any suitable software, firmware, and hardware for the above purposes. For example, one or more tablet PCs, smartphones, laptop computers, and the like may be included as part of mobile device 110. Mobile device 110 may be in communication with a separate display monitor, printer or other suitable peripheral devices.

To collect parameter data from the patient, one or more sensors 120 are configured to be placed on or near the patient for visual monitoring, audible monitoring, monitoring of physical characteristics, physiological conditions, other measurable characteristics or conditions, etc. Sensors 120 may include a temperature sensor, a pulse oximeter, a heart rate sensor, a breath rate sensor, a pulse rate sensor, a blood pressure sensor, glucomoter, a single lead electrocardiogram, a body weight sensor or other type of sensor suitable for collecting vital sign data from the patient. In an embodiment, a single sensor 120 is employed to obtain various types of patient parameters, such as heart rate, pulse rate, temperature and the like. In another embodiment, multiple sensors 120, each configured to obtain a different type of patient parameter, are used. Alternatively, one or more sensors 120 are configured to obtain measurements from the patient's bodily fluids, such as blood, urine, saliva, and the like. Sensors 120 are configured to obtain vital sign readings periodically (e.g., one reading every second), continuously or manually upon request. Sensors 120 transmit the collected parameter data either wirelessly or via a wired connection. Sensors 120 may include any suitable software, firmware, and/or hardware for the above-noted purposes.

Server 130 communicates with mobile device 110 for storing, processing, and/or transmitting information therebetween. More specifically, server 130 is configured to store information, e.g., the parameters, conditions, sensed data, reports, and/or other information, in a database and processes the information. Server 130 may include any suitable software, firmware, and hardware for these purposes and may establish the above-described communication via wired and/or wireless communication.

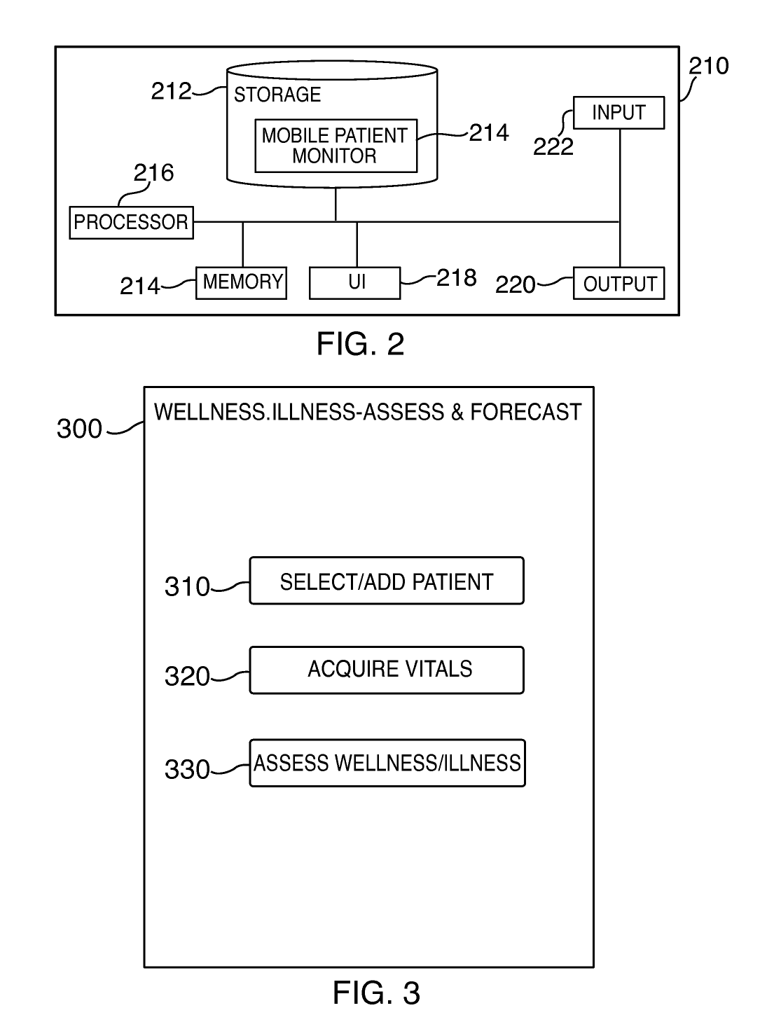

Turning now to FIG. 2, in conjunction with FIG. 1, one configuration of hardware and software components for receiving/transmitting information, e.g., control parameters, conditions, sensed data, and/or other information, processing the information, receiving user input, and/or displaying the information or otherwise outputting the information in accordance with the present disclosure is shown generally identified by system 200. System 200 generally includes a storage device 212, a memory 214, a processor 216, a user interface (UI) 218, an output 220, and an input 222, each of which may be embodied within or across one or more of mobile device 110, sensors 120, and server 130. That is, receiving/transmitting the information and user input, processing the information, and outputting the information for control, display, or other output may be performed locally, on one or more servers 130 for distribution to the mobile device 110, across a network, at mobile device 110, or in any combination of the above.

Storage device 212 may include any suitable component(s) operable for storing information received via input 222, such as, for example, a magnetic disk, flash memory, optical disk, or other suitable memory or data storage device. Memory 214 may include any computer memory, e.g., RAM or ROM, mass storage media, removable storage media, combinations thereof, or any other suitable computer-readable storage medium, storing instructions for causing processor 216 to execute particular functions, e.g., to process the information.

Processor 216 may include any suitable component(s), e.g., a central processing unit (CPU), operable to execute instructions stored in memory 214 to process and manipulate information, e.g., data stored in a database in storage device 212 or received via input 222, for output to UI 218 or output 220. Processor 216 is further configured to receive, via input 222 and/or UI 218, information, data, and/or control parameters for processing and manipulating the information in accordance with user-selected settings and user input.

UI 218 functions to output the processed information for visual display, e.g., in graphical and/or numerical form, to the user and/or allows for the input of information, data, setting of parameters, etc., by the user. In an exemplary embodiment, UI 218 is displayed on the display of mobile device 110. Output and input 222, 224, respectively, are provided to facilitate communication between system 200 and the other components of system 100 via wireless or wired communication. In an exemplary embodiment, input 224 is configured to receive information to be processed, e.g., data from sensors 120 (or other device where user-input data is provided), or input from the user, and the like.

With reference to FIGS. 3-6, display screens in accordance with embodiments of the present disclosure are shown displaying exemplary information as output by UI 218 to any of the displays described above. The display screens may be the only component initially displayed by UI 218 or may be one of many components displayed by UI 218 at the same time. According to an exemplary embodiment, the display screens provide the user with visual instructions to assess the patient's wellness or illness in an easy to understand format.

Turning now to FIG. 3, a welcome screen 300 is provided, in accordance with an exemplary embodiment. Welcome screen 300 includes buttons that allow the user to select one or more functions of the application to execute. Welcome screen 300 may include a “select/add patient” button 310, an “acquire vitals” button 320, and an “assess wellness/illness” button 330. The “select/add patient” button 310 launches a feature of the application that allows the user to select or add a patient's name. The “acquire vitals” button 320 launches a feature of the application that instructs sensors 120 to begin obtaining parameter data from the patient. In an exemplary embodiment, the “acquire vitals” button 320 also launches a feature that provides instructions on where to place sensors 120. The “assess wellness/illness” button 330 launches a feature of the application that assesses the wellness or illness of a selected patient based on the obtained information about the patient. As will be appreciated, welcome screen 300 may include one or more additional buttons for the selection of other features of the application, not shown in FIG. 3.

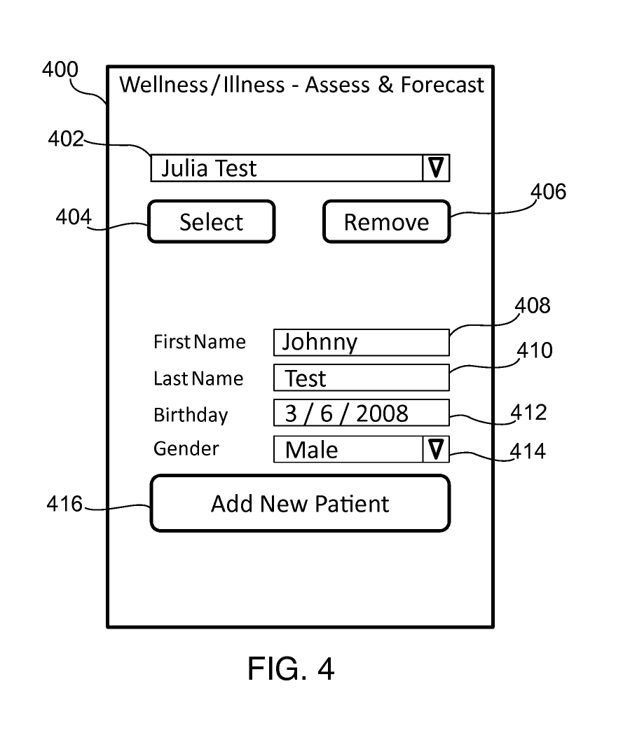

With reference to FIG. 4, a “select/add patient” screen 400 is depicted. The “select/add patient” screen 400 may be used to select a previously stored patient name or to input a new patient's name. For example, a pull-down menu 402 may be used to display one or more previously-added patient names from which the user may make a selection. Specifically, a patient name may be highlighted, and the user may select or remove the highlighted patient name by pressing a “select” button 404 or a “remove” button 406. Alternatively, the user may elect to add a new patient. In this regard, the user may enter the patient's first and last names into data fields 408, 410. If additional identifying information is desired, the user may input additional identifying information such as a birthdate or gender. For example, the birthdate may be added into a data field 412 and the gender may be selected via a pull-down menu 414. Once one or more of the data fields are completed, the user may select the “add new patient” button 416 and the new patient's information may be stored in a database.

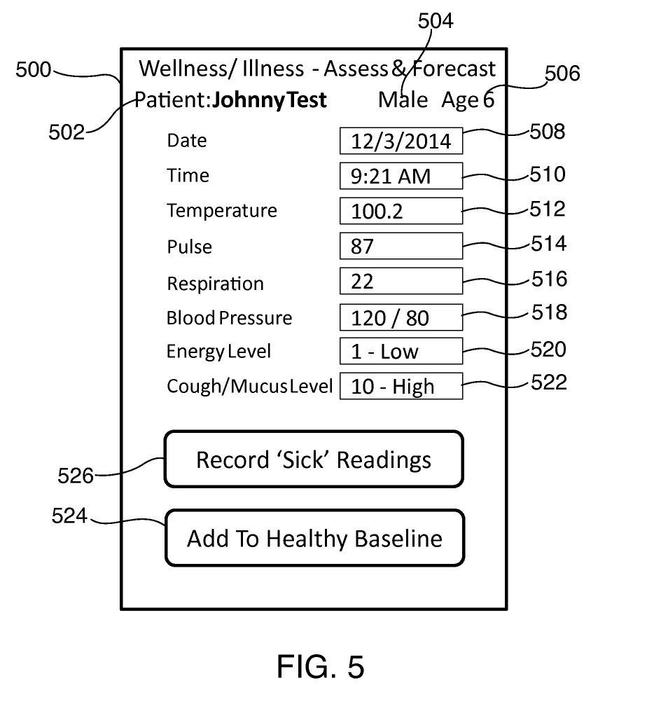

FIG. 5 illustrates a “patient vitals” screen 500 displaying, among other information, one or more of the parameters of the patient collected by sensors 120. The “patient vitals” screen 500 may include a patient name 502 and other patient identifying information. For example, the gender 504 and age 506 of the patient may be displayed. Additional information identifying the collection period, such as a date 508 and time 510 at which the parameter data were acquired from the patient may also be included. The “patient vitals” screen 500 may further display each particular parameter data obtained by sensors 120. Temperature 512, pulse 514, respiration 516, and blood pressure 518 may be included. In other embodiment of screen 500, additional patient parameter data, for example, measurements taken from blood, urine or other bodily fluids, may be included. Screen 500 also includes manual input fields 520, 522 for receiving manual input from the user. In particular, the manual input value may correspond to a subjective assessment of a particular condition of the patient and thus, may be a value that cannot be obtained from sensors 120. For example, the manual input fields 520, 522 may include categories such as energy level and/or cough and/or mucus level and the input values may be based on a number scale.

In an exemplary embodiment, the “energy level” field 520 may be used to indicate an energy level of the patient. The user may input a low value, such as “1.0,” if the user determines that the patient has a low amount of energy. Alternatively, the user may input a high value, such as “10.0,” if the user determines that the patient has a high amount of energy. Whether the patient has low or high energy may depend on whether the patient is usually energetic or not. For instance, if a normally active person lies in bed for an extended period of time, the user may enter a very low energy value into the “energy level” field 520. In another example, if a normally sedentary person is inactive, the user may enter a slightly low energy value. The “cough/mucus level” field 522 allows the user to manually input a value indicating a frequency or severity of the patient's cough. For example, if the patient exhibits a persistent dry cough, the user may input a lower value, for example, “5.0” while the user may choose to enter a “9.0” for a patient exhibiting a deep, loud, phlegmy cough. Alternative number scales may be utilized depending on the user's preference.

At the bottom portion of the “patient vitals” screen 500, the user may select different options for storing the acquired patient parameter data and manual input data. Screen 500 may include an “add to healthy baseline” button 524, as shown in FIG. 5, to allow the user to store acquired patient parameter data designated as healthy data. In an exemplary embodiment, the healthy data may be accumulated over time to create a historical database associated with the patient. The historical database may be used to derive baseline data of the patient. A “record ‘sick’ readings” button 526 may be included to allow the user to store the acquired patient parameter data as sick data. Sick data may be compared with the patient's baseline data as will be discussed later.

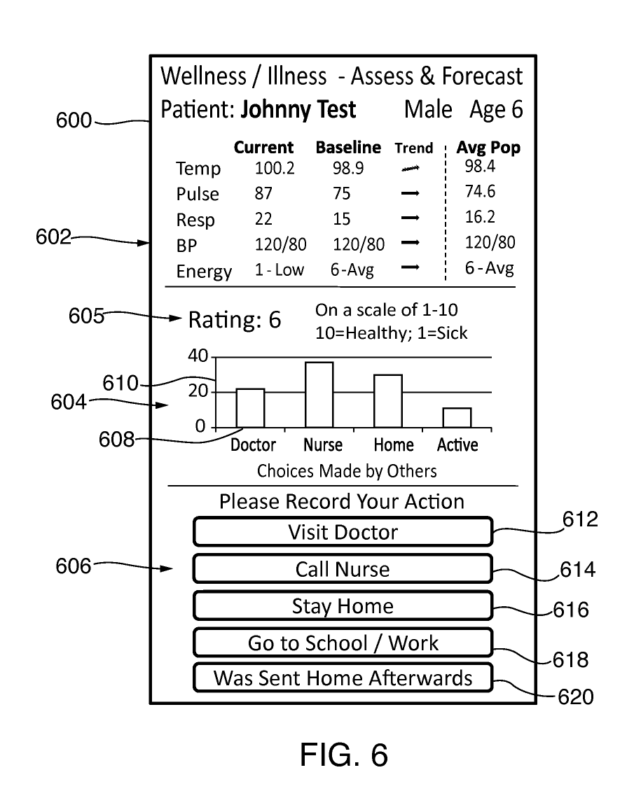

Referring to FIG. 6, an “assess wellness/illness” screen 600 is provided, displaying a concise summary of an overall status of the patient's condition and popular courses of action taken by others when faced with patient parameter and manual input data similar to those of the patient's. In an exemplary embodiment, the “assess wellness/illness” screen 600 includes a top section 602 providing the patient's identifying information and a summary of the patient's acquired vitals as compared to the patient's baseline data and data of other patients. As depicted in FIG. 6, one or more of the patient's acquired patient parameters may be listed under the heading “current” and may be included next to a column (“baseline”) showing corresponding baseline data of the patient. A “trend” column adjacent to the “baseline” column may be included to indicate whether the patient's current vitals are either as increasing, decreasing, or not changing as compared to the patient's baseline data. The trends may be represented by visual cues, such as arrows, flags or other symbol. The visual cues may be color-coded to show a severity of a trend. For instance, a red symbol may indicate a negative trend and a green symbol may indicate a positive trend. Color-coding may be implemented with the display of the “current” data. In an embodiment, a color shading of the values may be included with the display of each patient parameter.

At the far right of top section 602, an “average population” column may be included. The “average population” column includes the data of those patients who are of similar age and the same gender as the patient. In this way, the user may visually compare the current acquired patient parameters with those of a population including similarly situated patients. For example, in FIG. 6, the patient is a 6-year old male and hence, the data presented in the “average population” column may include average values derived from data obtained from other 6-year old males.

A middle section 604 of the “assess wellness/illness” screen 600 may include a rating 605 and a graph. Rating 605 may be a numerical or other value indicating sickness or a degree of sickness of the patient. For example, rating 605 may be a value on a scale of 1-10, where a patient in good condition may have a wellness rating that is near or at 10.0 and a patient in bad condition may have a wellness rating that is near or at 0.0. As shown in FIG. 6, rating 605 for the patient is a “6”, indicating that the patient is sick. The graph is included to show the popularity of courses of action taken by other users when patients under their care were found to have similar, or in an embodiment, identical parameters as the patient. In an exemplary embodiment, the graph is a bar graph with an X-axis 608 including a listing of the courses of action and a Y-axis 610 indicating the percentages of patients selecting the courses of action. Thus, for example, about 39% of users presented with a 6-year old male with patient parameters identical to those of patient “Johnny Test” called a nurse for further evaluation or care.

A bottom section 606 of the “assess wellness/illness” screen 600 may include action buttons (e.g., “visit doctor” button 612, “call nurse” button 614, “stay home” button 616, “go to school/work” button 618, and “was sent home afterwards” button 620). The action buttons allow the user to contribute his/her selected action to a central database. In an exemplary embodiment, the “visit doctor” button 612 is selected if the user decides to take the patient to the doctor. The “call nurse” button 614 is selected if the user decides to call a nurse or other health care professional for consultation. The “stay home” button 616 is selected if the user decides that to keep the patient at home. The “go to school/work” 618 button is selected if the user sends the patient to school or to work. In some cases, despite being well enough to attend school or go to work, the patient's condition may worsen throughout the day and the patient may be sent home. In such case, the user may return to the “assess wellness/illness” screen 600 and select the “was sent home afterwards” button 620.

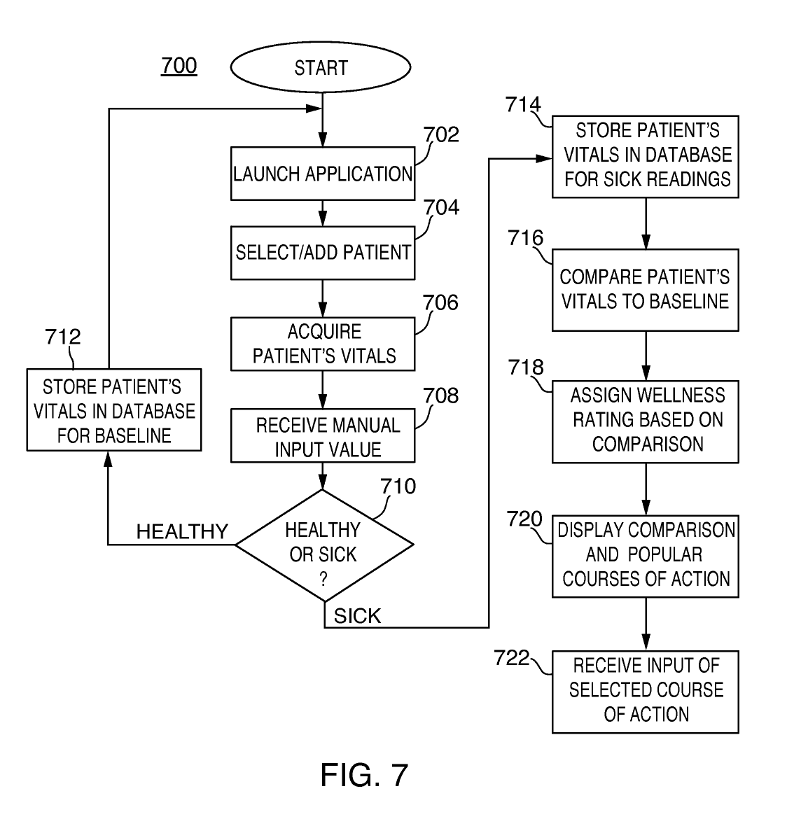

FIG. 7 is a flow diagram of a method 700 for assessing a wellness or illness of the patient, in accordance with an embodiment. Method 700 may be embodied in an application. In general, method 700 begins when the user launches the application at step 702. The application may be launched by receiving a manual input from the user on a user interface (e.g., UI 218), such as a touch, click or other input, on an icon displayed on the display of mobile device 110. In response to the input, the display may display an initial screen, such as welcome screen 300. A selection to select or add the patient is received at step 704. For example, the user may select or add a patient by selecting the “select/add patient” button 310, which may prompt the display of screen 400 to allow the user to select or add the patient as described above.

After the user either selects or adds the patient, a user input may be received instructing sensors to acquire parameter data from the patient at step 706. For example, the user may select the “acquire vitals” button 320, and in response to the selection, instructions are sent from mobile device 110 to sensors 120 instructing sensors 120 to obtain parameter data from the patient. As will be appreciated by those with skill in the art, sensors 120 may be placed on or adjacent the patient prior to or after the user input, and/or in contact with the patient's bodily fluid either in vivo or in a fluid collection receptacle, depending on the particular configuration of the sensor. As such, the patient parameter data may include vital sign data collected from the patient, measurement data from the bodily fluids of the patient, or other data collected from the patient.

As part of obtaining the patient parameter data manual input of an additional value corresponding to a subjective parameter of the patient may be optionally received at step 708. The user may visually assess the particular subjective parameter of the patient, for such as, an energy level or a cough frequency/severity of the patient, and may input an appropriate value into screen 500, for example. In an exemplary embodiment, only one of the subjective parameters is inputted. In another embodiment or one or more other subjective parameters may be included in addition or as an alternative to those previously mentioned.

At step 710, a user input is detected as to whether the patient parameter data and manual input data is healthy data, to indicate data obtained from the patient when the patient is known to be healthy, or sick data, to indicate data obtained from the patient when the patients is suspected as being sick. For example, the user may select the “record ‘sick’ readings” button 524 or the “add to healthy baseline” button 526. If the patient parameter and manual input data is designated as healthy data at step 710, the data is stored in a database with other healthy data at step 712. The other healthy data may include data previously obtained from the patient to create a historical database of the patient's parameter data. In an embodiment, the historical database is used to derive the patient's baseline data. For example, an average is maintained for each patient parameter to make up the patient's baseline data. It will be appreciated that because new patient parameter data is continuously being added to the database, the patient's baseline data may be dynamic. The healthy data may also be stored in a central database along with data collected from other patients. The data in the central database may be later used to determine corresponding gender/age baseline values. After the healthy data is stored, method 700 returns to step 702.

If the patient parameter and manual input data is designated as sick data at step 710, the data may be stored in another database at step 714. The sick data database may be a separate database from that of the healthy data database. In an exemplary embodiment, the sick data is stored in a database with sick data obtained from the patient during previous periods of time to create a historical database. In another exemplary embodiment, the sick data is sent to the central database, which stores the patient's sick data with patient parameter and manual input data added by other patients. The data stored in the central database may be used for purposes such as comparison of data of the patients, development of averages of the patient data based on various criteria, identification of health/sickness trends (e.g., spread of flu) among a certain population found in the patient data that can be used to generate alerts to users or to local, regional and national authorities. The data stored in the central database may be shared with insurance companies, incorporated into the patient's electronic medical record (if the central database is accessible by the patient's doctor's office or hospital), and the like.

Next, a user input to assess the patient's wellness/illness may be received, and in response to the input, the patient parameter data and manual input data are then compared to the patient's baseline data at step 716 and a wellness rating is assigned to the patient at step 718. The baseline data may be one or more values, such as integers, fractions, or a range or ranges of numbers, or letters derived from the healthy data. Alternatively, the baseline data may include averages of each patient parameter of the vital sign data stored as healthy data. In an embodiment in which the baseline data is a range, the ranges may be narrowed to provide relatively fine granularity between wellness ratings such that each range can correspond to a course of action, such as “call doctor,” “call nurse,” “stay home,” or “go to work or school.”

In an exemplary embodiment, a comparison is made by assigning values to the patient parameter data and manual input data and calculating a score using the assigned values. According to an exemplary embodiment, each patient parameter data and manual input data is scored against the baseline data, for example, either the baseline data of the patient or a baseline derived from data from other patients of the same gender and age. Alternatively, the patient parameters are scored against the gender/age baseline data and then offset by the patient's own baseline data. For example, in an embodiment in which the patient's baseline data for body temperature is 98.8° F. and the gender/age baseline data for body temperature is 98.6° F., 0.2° F. would be subtracted from the body temperature value prior to including the value in the score. In any case, the patient parameters may be scored against the baseline data in pre-set ranges. For example, a temperature of 98.6-100° F. may be assigned a score of “1.” In another exemplary embodiment, the patient parameters may be scored against the baseline data as standard deviations from the norm, where the norm is the baseline data. The patient parameter scores are then summed and compared against a predetermined threshold value or range.

In another exemplary embodiment, the score may be calculated by weighting one or more of the patient parameter and/or manual input data based on importance. For example, the patient parameters corresponding to a high body temperature or a fast pulse may be assigned a greater weight than an energy level. The weighting for one or more of the patient parameters and/or manual input data may also automatically increase as the presence of that data above or below a certain threshold increases in duration. In addition to or alternatively, the score may be calculated by pairing patient parameters and/or manual input data or combining three or more patient parameters and/or manual input data based on the covariance of their relationships. In an example, pulse rate and body temperature are related. Hence, when pulse rate and body temperature are scored individually, double-counting may occur. Thus, pulse rate and body temperature may be scored together to obtain a more accurate score. In yet another exemplary embodiment, paired or combined patient parameters and/or manual input data are scored based on a fuzzy-logic model, similar to the individual parameter scoring, except that the ranges and associated scores do not yield step functions and are more gradual. Other methods for calculating patient parameter scores are disclosed in U.S. patent application Ser. No. 13/768,769 filed on Feb. 15, 2013, U.S. patent application Ser. No. 13/942,019 filed on July 15, 2013, and U.S. patent application Ser. No. 14/287,729 filed on May 27, 2014, the entireties of which are incorporated herein by reference.

As noted above, the score is compared with a predetermined threshold. The score and predetermined threshold may be an integer, a fraction, range of numbers, or a letter useful for comparison. In an embodiment in which the predetermined threshold is a range, the score is compared with the range and a determination is made as to whether the score is within or outside of the range. In another embodiment in which the predetermined threshold is an integer, the comparison may include determining whether the score is above or below the integer.

In another embodiment, the patient parameter data and manual input data are not scored and are compared with the baseline value. For example, each of the obtained values of the patient parameter and manual input data are directly compared with corresponding baseline data. Standard deviations are calculated between each of the obtained values of the patient parameter data and manual input data and the baseline data, or a determination of whether the obtained values of the patient parameter data and manual input data fall within particularly defined ranges of the baseline data are made. In another example, weights are then assigned to each of the calculated values. In still another example, a determination is made based on a Scalar Vector Machine, k-Nearest Neighbor, or other machine learning technique where, based on actions of the population (or recommendations of doctors, nurses, and/or clinicians), a determination as to where the patient parameter data should fall.

Based on the comparison, a wellness rating is assigned to the patient, at step 718. The wellness rating is selected from a plurality of wellness ratings that span from a rating indicating a “healthy” patient to another rating indicating a “sick” patient for instance. To provide the user with a more accurate assessment of the patient's wellness, the wellness ratings may include three or five, or any other number of ratings to clearly convey to the user a seriousness of a patient's wellness or illness.

In an embodiment, the comparison is displayed as a table (for example, FIG. 6), one or more graphs, or another manner. In an example, the comparison is displayed as patient status information including the patient's wellness rating, which may be a number, where a patient in good condition may have a wellness rating that is near or at 10.0 and a patient in bad condition may have a wellness rating that is near or at 0.0. Alternative number scales may also be utilized depending on clinician and/or hospital preference and may be adjustable by the clinician. Alternatively, a color or other similar visual indicators or other forms of communicating the patient's wellness or illness may be employed. The patient state information may include trend indicators, for example, arrows indicating a positive or negative trend with regard to the patient's condition (for example, FIG. 6).

As briefly noted above, in addition to the comparison of step 716, another comparison may be made between the patient's parameter data and the manual input data with parameter data and manual input data collected from other patients. In this regard, a similar comparison as described in step 716 is performed and the outcome of the comparison may be conveyed to the user as either as part of a table (as shown in FIG. 6) or as a graph.

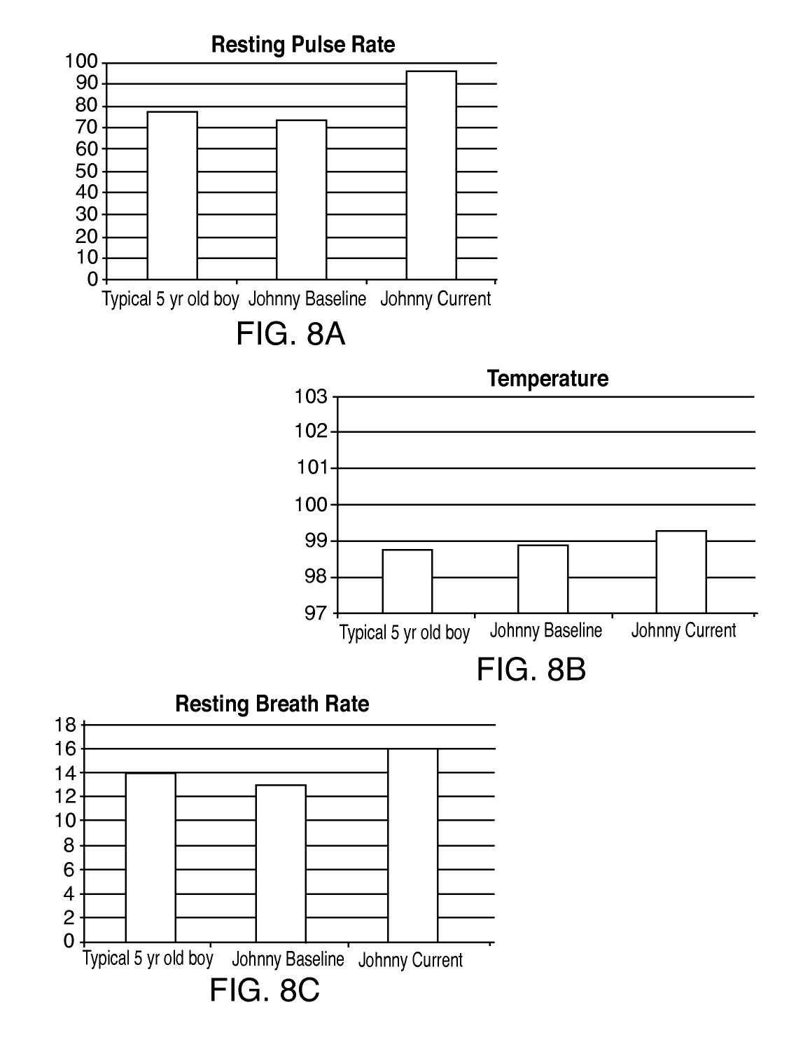

In another embodiment, both comparisons (for example, the comparison with the baseline data and the comparison with the similarly situated patients, are included in a single graph. FIGS. 8A-8C are bar graphs 800A, 800B, 800C including comparisons of resting pulse rate, temperature, and resting breath rate data from a 5-year old male “Johnny” with baseline data from other similar patients and baseline data from Johnny The comparison may be displayed as a bar graph, and color coding or visual cueing is used to highlight areas of concern, such as values that may be too high or too low. In another exemplary embodiment, a graph may be included showing a composite comparison based on both baseline data from Johnny and baseline data from other patients as compared to Johnny's current data.

After the wellness rating is assigned to the patient, popular courses of action corresponding to the wellness rating are displayed at step 720. For example, the patient's parameter data and the manual input data are compared with parameter data and manual input data collected from other similarly situated patients and matched with the courses of action selected by those other patients. According to an embodiment, the patient's wellness rating is compared with other patients' wellness ratings and corresponding courses of action taken by those patients with the same wellness rating are conveyed to the user. In an alternative embodiment, the patient's parameter data and manual input data is directly compared with the data of other similarly situated patients and the courses of action taken by those patients are then displayed. The courses of action and popularity of the courses of action may be displayed as a graph (as shown in FIG. 6), a table, or another form of communication. By providing a visual display of the courses of action taken according to popularity, the user may make a more informed and confident decision as to whether to send the patient to a doctor, nurse, school or work, or keep the patient at home.

After the user selects a course of action for the patient, a user input may be detected indicating the course of action selected by the user at step 722. In response, the selected course of action is stored in the database.

According to an exemplary embodiment, over time and repeated use of the application, a comprehensive database including the patient's and other patient parameter data and manual input data may be built to match a customized recommended course of action with the patient's current condition. To continuously improve the comprehensive database, the application may include reminder features, for example, calendared alerts, to prompt the user or patient to obtain patient parameter data from time to time. The alerts may be provided weekly, bi-weekly, monthly or more or less frequently. Other alerts may be provided to prompt the user or patient to obtain patient parameter data soon after any wellness rating other than a healthy rating, which may be useful for monitoring the patient's condition for improvement or deterioration.

The application may further include quick-reference database features that are associated with the “call doctor” button 612 or “call nurse” button 614 and allow the user to easily access phone or email information of the doctor or nurse, hospital information, insurance information, emergency contact information, 911, school or office contact information, and the like.

Certain embodiments of the present disclosure comprise logic for performing operations of the application described above, and may be embodied in at least one tangible, computer-readable medium. For example, when the logic is executed, it may be operable to receive data, determine scores from the received data, assign a wellness rating to the scores, and display courses of action based on the scores. In certain embodiments, the logic for performing operation of the application described above may be embodied in more than one tangible, computer-readable medium. For example, portions of the logic may be embodied in one or more of mobile device 110, sensors 120, or server 130 of system 100 in any manner.

While several embodiments of the disclosure have been shown in the drawings and described in detail hereinabove, it is not intended that the disclosure be limited thereto, as it is intended that the disclosure be as broad in scope as the art will allow. Therefore, the above description and appended drawings should not be construed as limiting, but merely as exemplifications of particular embodiments. Those skilled in the art will envision other modifications within the scope and spirit of the claims appended hereto.

What is claimed is:

1. A system for assessing a wellness or illness of a patient comprising:
a mobile device including a processor, a device memory in communication with the processor, and a display in communication with the processor, the device memory including instructions that, when executed, cause the processor to:
receive a plurality of patient parameters including one or more of a temperature of the patient, a pulse of the patient, a respiration rate of the patient, and a blood pressure of the patient;
receive a manual input of an additional value corresponding to a subjective parameter of the patient;
compare the plurality of patient parameters and the additional value with baseline data;
assign a wellness rating to the patient, based on the comparison; and
display courses of action associated with the wellness rating of the patient.

2. The system of claim 1, further comprising a server in communication with the mobile device, the server including a server memory storing one or more of the plurality of patient parameters, the wellness rating, and a patient identifier associated with the plurality of patient parameters in a database.

3. The system of claim 1, wherein the instructions further include instructions to display on the display a comparison of one of the patient parameters selected from the plurality of patient parameters with baseline data of the patient.

4. The system of claim 1, wherein the instructions further include instructions to display on the display a comparison of one of the patient parameters selected from the plurality of patient parameters with baseline data of other patients of the same gender and the same age.

5. The system of claim 1, wherein the instructions further include instructions to display a selection screen including a first course of action button and a second course of action button, the first course of action button being associated with a first wellness rating, and the second course of action button being associated with a second wellness rating.

6. The system of claim 1, further comprising a sensor in communication with the mobile device, the sensor configured to collect data from the patient related to the plurality of patient parameters and to send the collected data to the mobile device.

7. The system of claim 1, further comprising a plurality of sensors in communication with the mobile device, each sensor of the plurality of sensors configured to collect a type of patient parameter data that is different than a type of patient parameter data collected by another sensor of the plurality of sensors.

8. The system of claim 1, wherein the subjective parameter includes one or more of an energy level of the patient and a cough level of the patient.

9. A method of assessing a wellness or illness of a patient, comprising the steps of:
receiving a plurality of patient parameters including one or more of a temperature of the patient, a pulse of the patient, a respiration rate of the patient, and a blood pressure of the patient;
receiving a manual input of an additional value corresponding to a subjective parameter of the patient;
determining a score from the plurality of patient parameters and the additional value;
assigning a wellness rating to the patient, based on the determined score; and
displaying a course of action associated with the wellness rating of the patient.

10. The method of claim 9, wherein the assigning includes comparing the determined score to a predetermined value.

11. The method of claim 10, wherein the comparing of the determined score to the predetermined value includes analyzing a number of standard deviations the determined score is from baseline data.

12. The method of claim 9, wherein the determining further includes:
comparing one of the patient parameters selected from the plurality of patient parameters with baseline data of the patient.

13. The method of claim 9, wherein the baseline data includes sick data.

14. The method of claim 9, wherein the baseline data includes healthy data.

15. The method of claim 9, wherein the determining further includes:
comparing one of the patient parameters of the plurality of patient parameters with an average of a corresponding patient parameter obtained from other patients of the same gender and the same age.

16. A method of assessing a wellness or illness of a patient, comprising the steps of:
receiving a plurality of patient parameters including a temperature of the patient, a heart rate of the patient, and a blood pressure of the patient;
receiving a manual input of an additional value corresponding to a subjective parameter of the patient;
determining a score from the plurality of patient parameters and the additional value;
comparing the score with data obtained from a plurality of other patients of the same gender and the same age;
assigning a wellness rating to the patient, based on the comparison; and
displaying a plurality of courses of actions based on the wellness rating of the patient and based on the data obtained from the plurality of other patients of the same gender and the same age.

17. The method of claim 16, further comprising:
displaying a first course of action button and a second course of action button, the first course of action button being associated with a first wellness rating, and the second course of action button being associated with a second wellness rating.

18. The method of claim 17, further comprising:
receiving a selection of the first course of action button or the second course of action button; and
storing the received selection in a database.

19. The method of claim 18, wherein comparing further includes comparing the score with data obtained from the stored received selection in the database.

20. The method of claim 16, further comprising:
comparing one of the patient parameters selected from the plurality of patient parameters with baseline data of the patient.
